(19) United States
(12) Patent Application Publication
(10) Pub. No.: US 2012/0144686 A1
(43) Pub. Date: Jun. 14, 2012

(54) Wall Mountable Height Measuring Device

(76) Inventor: Zohar Haykeen, Valley Village, CA (US)

(21) Appl. No.: 12/968,162

(22) Filed: Dec. 14, 2010

(51) Int. Cl. A61B 5/107; G01B 1/00

(52) U.S. Cl. 33/512

(57) ABSTRACT

A wall mountable height measuring device to record development of height of a person is disclosed. The wall mountable height measuring device comprises a board, a pair of vertical scrolling bars, at least one linear measurement scale, a horizontal frame and a leveling bar. The horizontal frame includes a pair of sliding bars and a plurality of openings. The board includes a front surface, a rear surface, a left side and a right side. The pair of sliding bars includes a first sliding bar and a second sliding bar designed to slide between the pair of vertical scrolling bars. The plurality of openings is adaptable to hold at least one date stamp. The at least one date stamp indicates the date at which the height measurement was taken.

### CROSS-REFERENCE TO RELATED APPLICATIONS

Not Applicable.

### STATEMENT REGARDING FEDERALLY SPONSORED RESEARCH AND DEVELOPMENT

Not Applicable.

### FIELD OF THE INVENTION

This invention relates to height measuring devices, and more particularly to a wall mountable height measuring device for measuring and recording the changing height of a growing person.

### DISCUSSION OF RELATED ART

Height is an important measurement for assessing the fitness of a person. A number of traditional height measuring devices have been developed in the art. These devices help the person to record progressive increases in height. Most conventional height measuring systems adopt a method in which, a person stands against a wall and the person's height is marked on the wall. Such markings will fade over time and become lost.

One prior art, U.S. Pat. No. 7,059,060 issued to Baumgartner describes a device for recording both chronological events and physical growth events of an individual or group of individuals. The device includes a linear measuring device having at least first and second opposed sides. The first side has a linear measurement scale inscribed thereon and at least one recording surface associated therewith. The second side has a chronological scale, preferably in months and years, inscribed thereon and also has at least one recording surface associated with the chronological scale. Thus, physical growth can be measured on the first side and recorded on the recording surface associated therewith as the individual or several individuals grow. Significant events such as the date of height measurement can be recorded on the other side. However, this device has two separate sides for measuring height and date and both measurements cannot be recorded simultaneously. Further, the device is not wall mountable.

Another prior art, U.S. Pat. No. 6,226,881 issued to Landauer describes a height-measuring device that is foldable, or collapsible, on itself, in order to provide an easier and less-costly method of packaging and shipping. The height measuring device consists of an extensible leg or setup section that is mounted in the rear of the scale-part. The extensible leg has a length equal to the lowest measurement reading of the scale-section. The bottom edge surface of the scale-part can be readily located during installation of the height measuring device. However, the device has no arrangement to record the date at which the height measurement was taken.

U.S. Pat. No. 4,196,521 issued to Hutchinson describes a height measuring device that includes telescopically arranged measuring rods with faces containing parallel vertical columns of different types of height measuring units such as English measuring units and metric measuring units. However, this height measuring device is quite heavy and hence not portable.

Therefore, there is a need for an improved height measuring device that would accurately measure the changing height of a growing person. Such a needed device would have an arrangement to simultaneously record the height and date at which the measurement was taken. Further, such a device would be portable and wall mountable. The present invention accomplishes these objectives.

### SUMMARY OF THE INVENTION

The present invention is a wall mountable height measuring device to record development of height of a person. The wall mountable height measuring device comprises a board, a pair of vertical scrolling bars, at least one linear measurement scale, a horizontal frame and a leveling bar. The horizontal frame includes a pair of sliding bars and a plurality of openings. The board includes a front surface, a rear surface, a left side and a right side. One of the pair of vertical scrolling bars is attached on the left side and other one of the pair of vertical scrolling bars is attached on the right side. The pair of sliding bars includes a first sliding bar attached with a first side of the horizontal frame and a second sliding bar attached with a second side of the horizontal frame. The first sliding bar and the second sliding bar are designed to slide between the pair of vertical scrolling bars. The plurality of openings is adaptable to hold at least one date stamp. The at least one date stamp indicates the date at which the height measurement was taken. The leveling bar may be attached at the center of the horizontal frame and may be adaptable to place over head of a person. The wall mountable height measuring device may be mounted on a wall by means of at least one attachment means.

The present invention facilitates an efficient way for accurately measuring the changing height of a growing person. Such a device has an arrangement to simultaneously record the height and date at which the measurement was taken. Further, such a device is portable and wall mountable. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is a perspective view of the present invention, illustrating a wall mountable height measuring device;

FIG. 2 is a perspective view of another embodiment of the wall mountable height measuring device; and

FIG. 3 is a perspective view of the invention, illustrating the wall mountable height measuring device in use.

### DETAILED DESCRIPTION OF THE PREFERRED EMBODIMENT

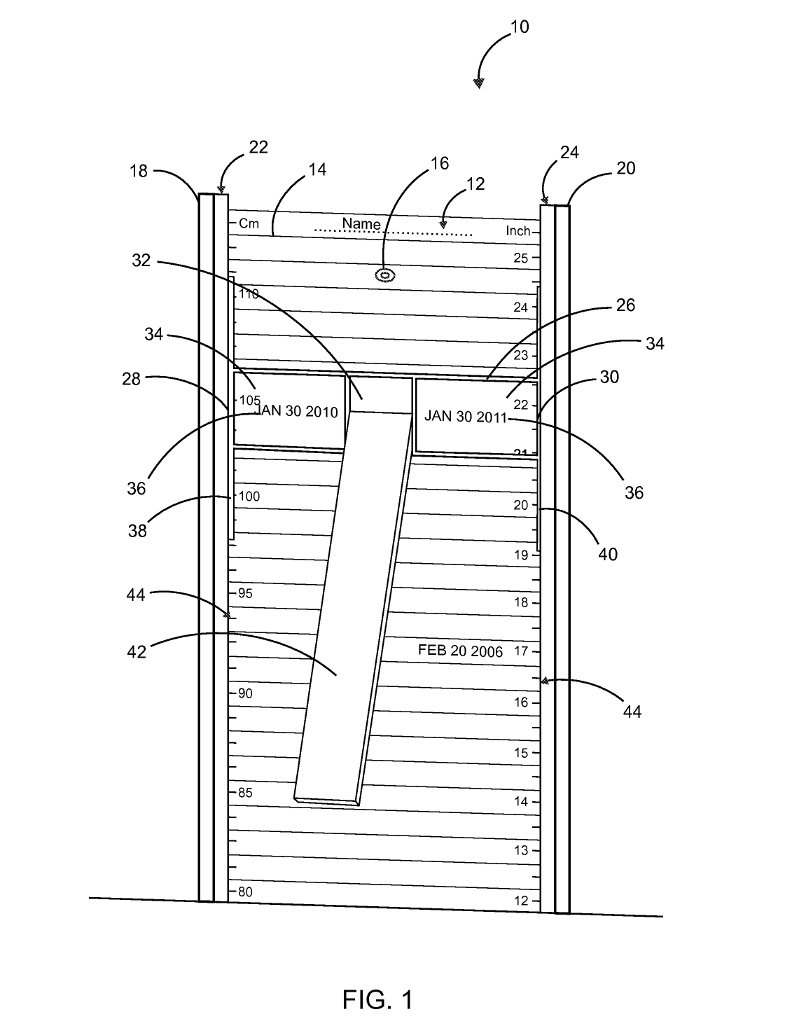

FIG. 1 is a perspective view of a wall mountable height measuring device 10 to record development of height of a person (not shown). The wall mountable height measuring device 10 comprises a board 12, a pair of vertical scrolling bars 22, 24, at least one linear measurement scale 44, a horizontal frame 26 and a leveling bar 42. The horizontal frame 26 includes a pair of sliding bars 38, 40 and a plurality of openings 34. The board 12 includes a front surface 14, a rear surface (not shown), a left side 18 and a right side 20. One of the pair of vertical scrolling bars 22 is attached on the left side 18 and other one of the pair of vertical scrolling bars 24 is attached on the right side 20. The pair of sliding bars 38, 40 includes a first sliding bar 38 attached with a first side 28 of the horizontal frame 26 and a second sliding bar 40 attached with a second side 30 of the horizontal frame 26. The first sliding bar 38 and the second sliding bar 40 are designed to slide between the pair of vertical scrolling bars 22, 24. The plurality of openings 34 is adaptable to hold at least one date stamp 36. The at least one date stamp 36 indicates the date at which the height measurement was taken. The leveling bar 42 may be attached at the center 32 of the horizontal frame 26 and may be adaptable to place over head of a person (not shown). The wall mountable height measuring device 10 is portable. The wall mountable height measuring device 10 may be mounted on a wall by means of at least one attachment means 16. The at least one attachment means 16 may be selected from a group consisting of screws, bolts and clamps.

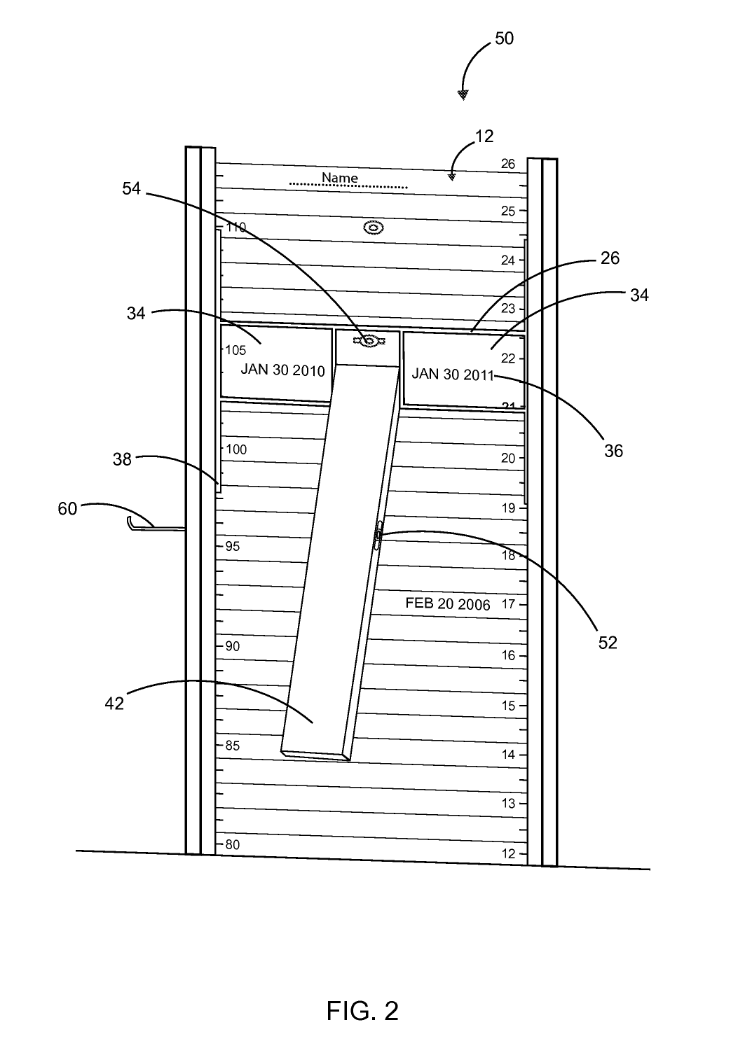

FIG. 2 is a perspective view of another embodiment 50 of the wall mountable height measuring device 10 of the present invention. The leveling bar 42 of the wall mountable height measuring device 50 may have a bubble 52 in liquid to adjust the position of the leveling bar 42. To prevent movement of the horizontal bar, once it is in its accurate position, the horizontal frame 26 may be attached to the board 12 by means of at least one fastening means 54. The at least one fastening means 54 may be a screw. The at least one date stamp 36 may be inserted into the plurality of openings 34 utilizing at least one stamping device (not shown). The wall mountable height measuring device 50 may include at least one anchoring means 60 to hold the at least one stamping device (not shown).

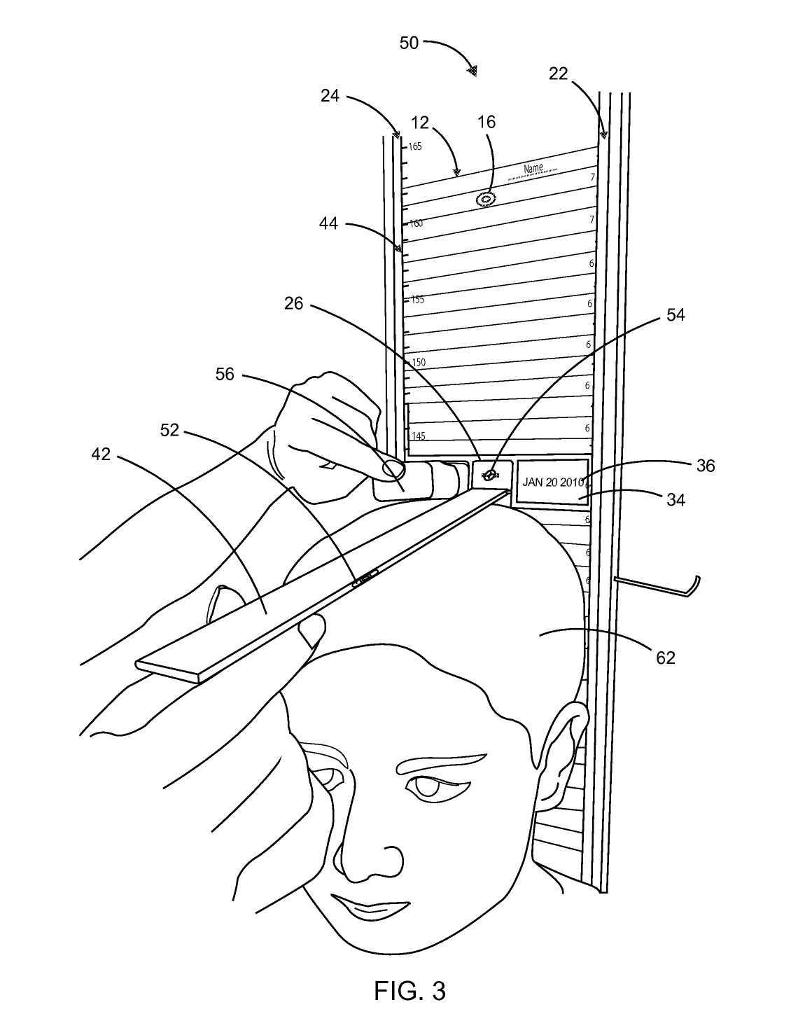

FIG. 3 is a perspective view of the invention, illustrating the wall mountable height measuring device 50 in use. While in use, the wall mountable height measuring device 50 is mounted on the wall by means of the at least one attachment means 16 and the person 62 is positioned against the board 12. The horizontal frame 26 is slided between the pair of vertical scrolling bars 22, 24 towards the head of the person 62. The leveling bar 42 is placed over the head of the person 62 and the position of the levelling bar 42 is adjusted using the bubble 52. When the bubble 52 shows an accurate level of the leveling bar 42, the horizontal frame 26 is attached to the board 12 by means of the at least one fastening means 54. The accurate measurement of the height is taken from the at least one linear measurement scale 44. The at least one date stamp 36 is inserted into one of the plurality of openings 34 utilizing the at least one stamping device 56.

While a particular form of the invention has been illustrated and described, it will be apparent that various modifications can be made without departing from the spirit and scope of the invention. Accordingly, it is not intended that the invention be limited, except as by the appended claims.

What is claimed is:

1. A wall mountable height measuring device comprising:
a board having a front surface, a rear surface, a left side and a right side;
a pair of vertical scrolling bars, one of the pair of vertical scrolling bars attached on the left side and other one of the pair of vertical scrolling bars attached on the right side;
at least one linear measurement scale attached on the left side and the right side;
a horizontal frame having a pair of sliding bars and a plurality of openings, the pair of sliding bars being designed to slide between the pair of vertical scrolling bars; and
a leveling bar attached to a middle portion of the horizontal frame;
whereby the plurality of openings is designed to hold at least one date stamp to record development of height of a person.

2. The wall mountable height measuring device of claim 1 wherein the leveling bar may be adaptable to place over head of the person.

3. The wall mountable height measuring device of claim 1 wherein the leveling bar may have a bubble in liquid to adjust the position of the leveling bar.

4. The wall mountable height measuring device of claim 1 wherein the pair of sliding bars includes a first sliding bar and a second sliding bar.

5. The wall mountable height measuring device of claim 4 wherein the first sliding bar is attached with a first side of the horizontal frame and the second sliding bar is attached with a second side of the horizontal frame.

6. The wall mountable height measuring device of claim 1 wherein the horizontal frame may be attached to the board by means of at least one fastening means.

7. The wall mountable height measuring device of claim 1 wherein the at least one fastening means may be a screw.

8. The wall mountable height measuring device of claim 1 wherein the device may be mounted on a wall by means of at least one attachment means.

9. The wall mountable height measuring device of claim 1 wherein the at least one attachment means may be selected from a group consisting of screws, bolts and clamps.

10. The wall mountable height measuring device of claim 1 wherein the at least one date stamp may be inserted into the plurality of openings utilizing at least one stamping device.

11. The wall mountable height measuring device of claim 1 wherein the device may include at least one anchoring means to hold the at least one stamping device.

12. The wall mountable height measuring device of claim 1 wherein the height measuring device is portable.

13. The wall mountable height measuring device of claim 1 wherein the at least one linear measurement scale may be utilized to measure the height in feet/inches and in centimetres.

14. A method for measuring and recording height of a person comprising:
a) providing a wall mountable height measuring device;
b) mounting the wall mountable height measuring device on a wall;
c) positioning a person against a board;
d) sliding a horizontal frame between a pair of vertical scrolling bars towards head of the person;
e) placing a leveling bar over the head of the person;
f) taking an accurate measurement from at least one linear measurement scale on the board; and
g) inserting at least one date stamp into one of a plurality of openings in the horizontal frame.

15. The method of claim 14 wherein the at least one date stamp indicates date at which the height measurement was taken.

16. The method of claim 14 wherein the wall mountable height measuring device may be mounted on a wall by means of at least one attachment means.
